(19) United States
(12) Patent Application Publication
(10) Pub. No.: US 2016/0166297 A1
(43) Pub. Date: Jun. 16, 2016

(54) BONE PLATE WITH ELEVATED SUTURE HOLE STRUCTURES

(71) Applicants: Mark Alan Mighell, Tampa, FL (US); Sergio Gutierrez, Tampa, FL (US)

(72) Inventors: Mark Alan Mighell, Tampa, FL (US); Sergio Gutierrez, Tampa, FL (US)

(73) Assignee: EXTREMITY DESIGNS, LLC, Tampa, FL (US)

(21) Appl. No.: 14/754,825

(22) Filed: Jun. 30, 2015

Related U.S. Application Data
(63) Continuation-in-part of application No. 14/569,401, filed on Dec. 12, 2014.

(51) Int. Cl. A61B 17/80; A61F 2/40; A61F 2/32; A61F 2/38; A61B 17/04; A61F 2/30

(52) U.S. Cl. CPC A61B 17/8057; A61B 17/80; A61B 17/8061; A61B 17/0401; A61B 17/8019; A61F 2/30; A61F 2/32; A61F 2/38; A61F 2/40; A61B 2017/00004

(57) ABSTRACT

The present invention includes a bone fixation plate assembly comprising a bone plate and a plate compression device for internal fixation of bone and tissue. The bone plate includes a top surface and a bottom bone-facing surface, one or more fastener holes extending between the top surface and the bottom bone-facing surface for fasteners, and one or more suture cleats extending from at least a portion of a boundary of the bone plate and configured to have one or more sutures passed therethrough. In one embodiment, the bone plate has a suture cleat configured to be elevated above the bottom bone-facing surface of the bone plate by a distance of between about 1-5 mm for providing suturing clearance for a suturing instrument, for example.

### CROSS-REFERENCE TO RELATED APPLICATIONS

The present application is a continuation-in-part of U.S. patent application Ser. No. 14/569,401, filed on Dec. 12, 2014, and entitled “Bone Plate with Elevated Suture Hole Structures” which claims the benefit of U.S. provisional patent application Ser. No. 61/712,257, filed Dec. 12, 2013, and entitled “Humeral Fracture Plate with Suture Hole Projections”. All of the foregoing applications are incorporated herein by reference in their entireties.

### FIELD OF INVENTION

This invention relates to a bone plate for use in repairing bone fractures.

### BACKGROUND OF THE INVENTION

Proximal humerus fractures are most commonly repaired with open reduction and internal fixation using plates and screws attached via bi-cortical or uni-cortical fixation. The preferred method to gain access to the fracture site is by making a large incision through the skin and muscles. Once the fracture has been exposed, the fragments of bone are approximated to the plate, including fragments that are attached to muscles via tendons. These muscles (e.g., rotator cuff) are attached to the plate via suture holes designed into it. Problems arise when the plate is first attached to the bone since it is difficult to pass the sutures between the plate and the bone. Thus, manufactures have provided bone plates with suture holes including undulations or suture-clearance recesses or lateral channels formed into the bottom surface and the edge of the plate and placed in relative proximity with corresponding suture holes such that a straight or curved suture needle and attached suture material may be passed through the hole even when the plate is fixed to the bone. This solution still presents challenges during surgery as there is insufficient space or clearance between the bone plate and the bone. Additionally, the superior screws often have the problem of exiting the humeral head superiorly, thus these screws must be able to be adjusted inferiorly to be directed completely into the humeral head.

The present invention seeks to remedy these problems. The object of the invention is to provide an internal fixation system with a plate which provides the surgeon with flexibility, ease of use, and operational efficiency such that a suture can be easily and quickly passed through a suture hole.

Another object of the invention is to provide a bone plate that supports both unidirectional and surgeon-directed or omnidirectional fixation of the screws relative to the plate.

### SUMMARY OF INVENTION

This invention achieves the objective with a bone plate having a first upper surface and a first opposed bone-facing surface, the first bone-facing surface shaped to generally conform to a plate-facing surface of the bone, a bone plate thickness, at least one fastener hole extending between the first upper surface and the first bone-facing surface, and a suture hole structure extending from at least a portion of a boundary of the bone plate and having a second upper surface and a second opposed bone-facing surface, a suture hole structure thickness, at least one suture hole extending between the second upper surface and the second bone-facing surface, wherein the suture hole structure thickness is less than the bone plate thickness, and the second bone-facing surface of the suture hole structure is elevated above the first bone-facing surface of the bone plate by a distance greater than 0 mm.

In another embodiment of the invention, the second upper surface of the suture hole structure is flush with and, optionally, has a substantially similar contour as the contour of that portion of the first upper surface of the bone plate where the suture hole structure extends from.

In accord with another embodiment, the fastener hole of the bone plate comprises two or more sets of threads with intersecting axes, wherein the angle of each axis is predetermined during manufacturing.

In a further development of the invention, the intersecting axes of sets of threads of the fastener hole lie in a plane substantially parallel to at least one of a longitudinal plane which divides the plate into left and right portions and a transverse plane which divides the bone plate into proximal and distal portions.

Various bone fasteners, such as screws and pegs, can be used with the current invention, for example, those with partially spherical or conical heads with or without external threads engageable with the threads on the inner wall surface of the fastener hole.

These and other features of various embodiments can be understood from a review of the following detailed description in conjunction with the accompanying drawings.

It is to be understood that both the foregoing general description and the following detailed description and accompanying drawings are exemplary and explanatory and are not restrictive of the present invention, as claimed.

### BRIEF DESCRIPTION OF THE DRAWINGS

The present invention and its developments will become more fully understood from, but not limited by, the detailed description and the accompanying drawings, wherein:

FIG. 1A shows a top view of a bone plate with elevated suture hole structures;

FIG. 1B shows a bottom view of the bone plate according to FIG. 1A;

FIG. 1C shows a side view of the bone plate according to FIG. 1A;

FIG. 1D shows another side view of the bone plate according to FIG. 1A;

FIG. 1E shows an end view of the bone plate according to FIG. 1A;

FIG. 1F shows another end view of the bone plate according to FIG. 1A;

FIG. 2A shows a top perspective view of a bone plate with the bone screws inserted and the top two screws angled superiorly;

FIG. 2B shows a bottom perspective view of the bone plate according to FIG. 2A;

FIG. 2C shows another bottom perspective view of the bone plate according to FIG. 2A;

FIG. 2D shows a side perspective view of the bone plate according to FIG. 2A;

FIG. 2E shows an end perspective view of the bone plate according to FIG. 2A;

FIG. 3A shows a top perspective view of the bone plate according to FIG. 2A with the top two screws angled inferiorly;

FIG. 3B shows a bottom perspective view of the bone plate according to FIG. 3A;

FIG. 3C shows another bottom perspective view of the bone plate according to FIG. 3A;

FIG. 3D shows a side perspective view of the bone plate according to FIG. 3A;

FIG. 3E shows an end perspective view of the bone plate according to FIG. 3A;

FIG. 4A shows a cross sectional view of the bone plate shown in FIG. 3A taken along sectional line 4A-4A of FIG. 3A;

FIG. 4B shows an alternative embodiment of the threaded hole shown in FIG. 4A; and

FIG. 5 shows a cross sectional view of the bone plate shown in FIG. 3A taken along sectional line 5-5 of FIG. 3A.

FIG. 6A shows a side perspective view of the proximal portion of a humeral plate with suture cleats as implanted on a humerus (not shown);

FIG. 6B shows another side perspective view of the bone plate shown in FIG. 6A;

FIG. 7A shows a top perspective view of the bone plate shown in FIG. 6A with a mounted plate compression device;

FIG. 7B shows a cross sectional view of the bone plate and the mounted plate compression device shown in FIG. 7A taken along sectional line 7B-7B of FIG. 7A; and

FIG. 7C shows a top perspective view of the bone plate and the mounted plate compression device shown in FIG. 7A.

### DETAILED DESCRIPTION OF THE PREFERRED EMBODIMENT

The following detailed description and the appended drawings describe and illustrate various bone plate systems, methods, and components. The description and drawings are exemplary in nature and are provided to enable one skilled in the art to make and use one or more exemplary bone plate systems and/or components, and/or practice one or more exemplary methods. They are not intended to limit the scope of the claims in any manner.

The use of “e.g.,” “etc.,” “for instance,” “in example,” and “or” and grammatically related terms indicates non-exclusive alternatives without limitation, unless otherwise noted. The use of “optionally” and grammatically related terms means that the subsequently described element, event, feature, or circumstance may or may not be present/occur, and that the description includes instances where said element, event, feature, or circumstance occurs and instances where it does not. The use of “exemplary” refers to “an example of” and is not intended to convey a meaning of an ideal or preferred embodiment. The use of “attached” and “coupled” grammatically related terms refers to the fixed, releasable, or integrated association of two or more elements and/or devices with or without one or more other elements in between. Thus, the term “attached” or “coupled” and grammatically related terms includes releasably attaching or fixedly attaching two or more elements and/or devices in the present or absence of one or more other elements in between. As used herein, the terms “proximal” and “distal” are used to describe opposing axial ends of the particular elements or features being described in relation to anatomical placement.

While the systems, methods, and components described herein are exemplified by systems and methods for internal fixation of humeral bones, the systems, methods, and components described and illustrated herein can be used to treat any short and long bones within the body of a human, including, but not limited to, animals. Skilled artisans will be able to select a suitable ailment and/or bone within the body of an animal to utilize a system and/or method described herein according to a particular embodiment based on various considerations, including the type of ailment and/or the structural arrangement at a treatment site. Example bones considered suitable to utilize a system, method, and/or component described herein include, but are not limited to, humerus, ulna, radius, clavicle, femur, tibia, fibula, tarsals, metatarsals, carpals, metacarpals and phalanges.

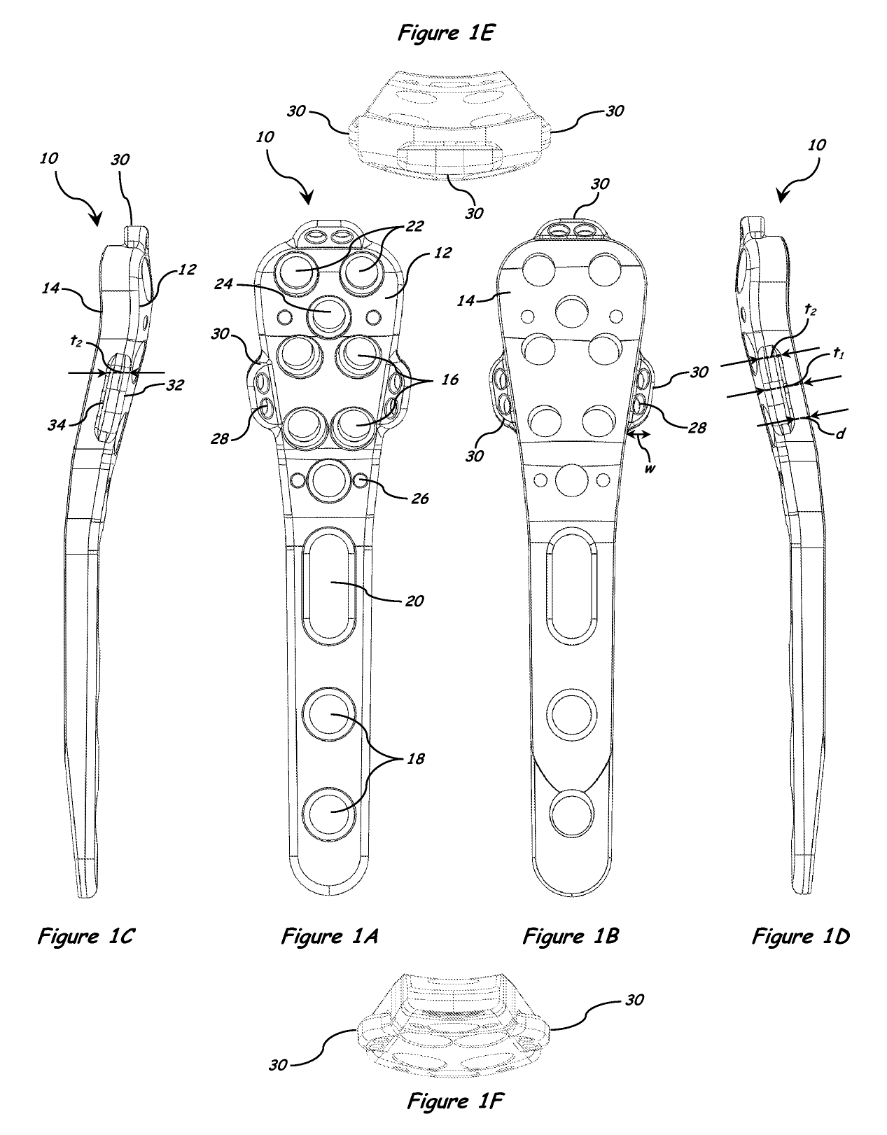

FIGS. 1A-F shows a bone plate configuration in accordance with the invention. Bone plate 10 may be shaped and configured for, but not limited to, fractures of the humerus. The bone plate 10 includes an upper surface 12, a lower or opposed bone-facing or bone-contacting surface 14 and a plurality of fastener holes 16, 18, 20, 22, 24 and k-wire holes 26 extending between the upper surface 12 and the opposed bone-facing surface 14 for receiving corresponding bone fasteners (not shown) and guide-wires (not shown) respectively. The bone-facing surface 14 can be shaped to substantially conform to or mate with a corresponding plate-facing surface of the bone (not shown) and may be provided with radiused or scalloped cutouts between fastener holes to limit and/or minimize contact between the bone-facing surface 14 and the bone. Limiting and/or minimizing contact between the bone plate 10 and bone has a number of biological and mechanical advantages including reduced damage to blood supply and easier plate removal. Bone plate 10 may have various sizes (various diameters and/or lengths) and may be constructed from biocompatible materials such as titanium, alloys of titanium, cobalt chrome, stainless steel, ceramics, composite materials such as carbon fiber-reinforced PEEK, resorbable materials, and combinations thereof, although one of ordinary skill in the art will know and appreciate that any biocompatible material may be used.

Referring now to FIGS. 1C-D, the upper surface 12 and the opposed bone-facing surface 14 run substantially parallel defining a first nominal thickness “t₁” of the bone plate 10. The bone plate 10 further includes a plurality of elevated suture hole projections or structures 30 each extending from at least a portion of a boundary or edge portion of the bone plate 10. Each suture hole structure 30 includes an upper surface 32 and an opposed bone-facing surface 34 defining a second nominal thickness “t₂” of the suture hole structure 30 wherein the thickness “t₂” may be less than the thickness “t₁” of the bone plate 10, and the bone-facing surface 34 or at least a portion thereof of the suture hole structure 30 is elevated above the bone-facing surface 14 of the bone plate 10 by a distance “d” as shown in FIGS. 1C-D. The distance “d” may be in the range of about 1-4 mm. Alternatively, the distance “d” can be lesser or greater, depending on a specific surgical application.

The suture hole structures 30 can be dimensioned and configured to provide a low profile for reducing soft tissue irritation and minimizing patient discomfort. The suture hole structure 30 may have a width “w” of about 1-4 mm. The width “w” may also be lesser or greater, depending on a specific surgical application. The upper surface 32 and the lower bone-facing surface 34 of the suture hole structure 30 may be substantially parallel, at an angle relative to one another, or tapered inwardly and/or outwardly along its length or width. The upper surface 32 and the lower bone-facing surface 34 of the suture hole structure 30 each may also have a convex or concave shape, or a combination thereof.

The suture hole structures 30 each comprises a plurality of suture holes 28 extending between the upper surface 32 and the bone-facing surface 34. The suture hole structure 30 may be provided with any number of suture holes 28 as may be suitable for a specific surgical application. Alternatively, the suture hole structure 30 may have only one suture hole 28. The shape of the suture holes 28 can be circular, oval or non-circular. The suture holes 28 can be of a size adequate for passing a suture with a curved or straight suture needle and can be non-threaded for reducing suture damage.

The upper surface 32 of the suture hole structure 30 may be flush with the upper surface 12 of the bone plate 10. Alternatively, the upper surface 32 of the suture hole structure 30 can be slightly higher or lower than the upper surface 12 of the bone plate 10 and/or can have a substantially similar contour to a contour of the portion of the boundary or the edge portion of the bone plate 10 from which the suture hole structure 30 extends. The suture hole structure 30 may be at an angle with respective to the bone plate 10. The suture hole structure 30 may have at least a portion of its body being as thick or thicker than the bone plate 10 as long as at least a portion of the bone-facing surface 34 of the suture hole structure 30 is elevated above the bone-facing surface 14 of the bone plate 10 to provide a suture-clearance or spacing “d” for easy access to the suture holes 28.

The suture hole structure 30 can be permanently or removably attached or coupled to the bone plate 10 by any attachment means known to one skilled in the art. The suture hole structures 30 and the bone plate 10 can be an unitary device machined from a single block of materials, or can also be a multi-component device which can be assembled before or during surgery to provide the surgeon the flexibility in designing the bone plate to meet his or her needs.

An alternative embodiment of the present invention (not shown) includes a bone plate substantially similar to the bone plate 10 in FIGS. 1A-F, wherein the suture hole structure or structures may be folded toward the bone to provide a low profile implant. One method of manufacturing such an implant is to have the portion of the suture hole structure proximate the boundary of the bone plate be thinner than the remaining part of the suture hole structure to allow the surgeon to bend or fold the suture hole structure toward the bone after passing a suture for reducing soft tissue irritation and minimizing patient discomfort. Another design may require a hinged mechanism for coupling the suture hole structure to the perimeter of the bone plate.

Still another embodiment of the invention (not shown) comprises a bone plate substantially similar to the bone plate 10 in FIGS. 1A-F, wherein the bone plate is provided with a rail extending along its perimeter or boundary. One of more suture hole structures similar to the suture hole structure 30 are coupled to the rail. This configuration provides the surgeon the flexibility to reposition the suture hole structure(s) anywhere on or along the bone plate to meet his or her specific surgical applications and needs.

According to another embodiment, the bone plate 10 can be provided with a single continuous suture hole structure (not shown) surrounding the boundary of the bone plate 10. This feature provides the surgeon the flexibility of attaching any muscles associated with the fractured bone to any locations on the plate to meet his or her needs for a specific surgical application.

These and other similar variations and modifications may be made without departing from the scope of the present invention.

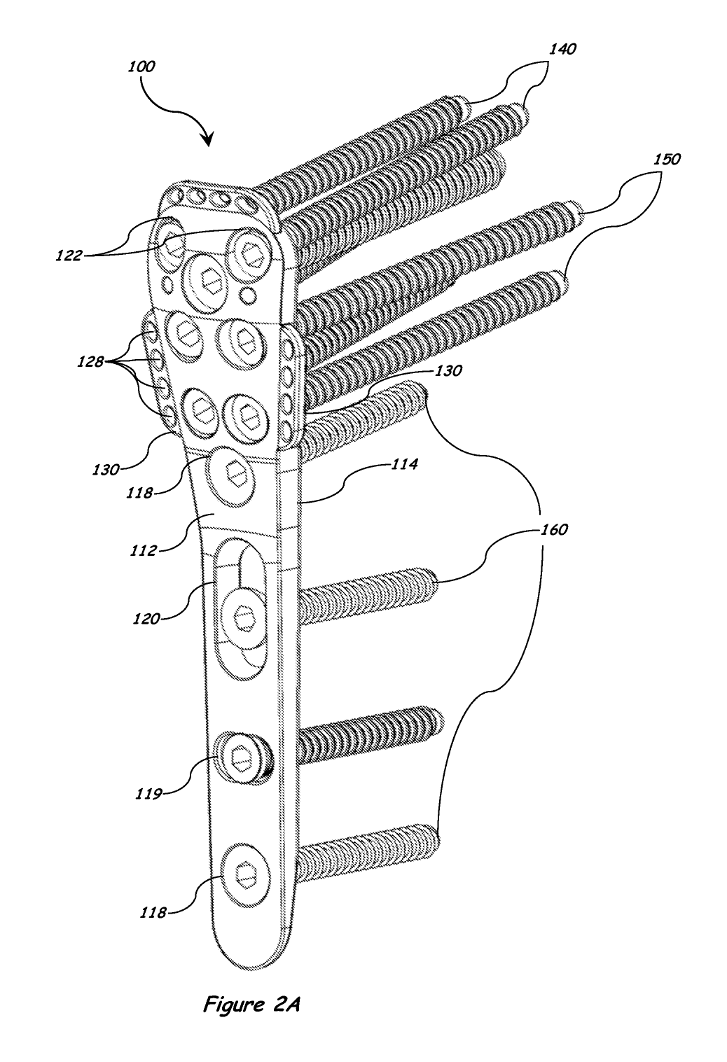

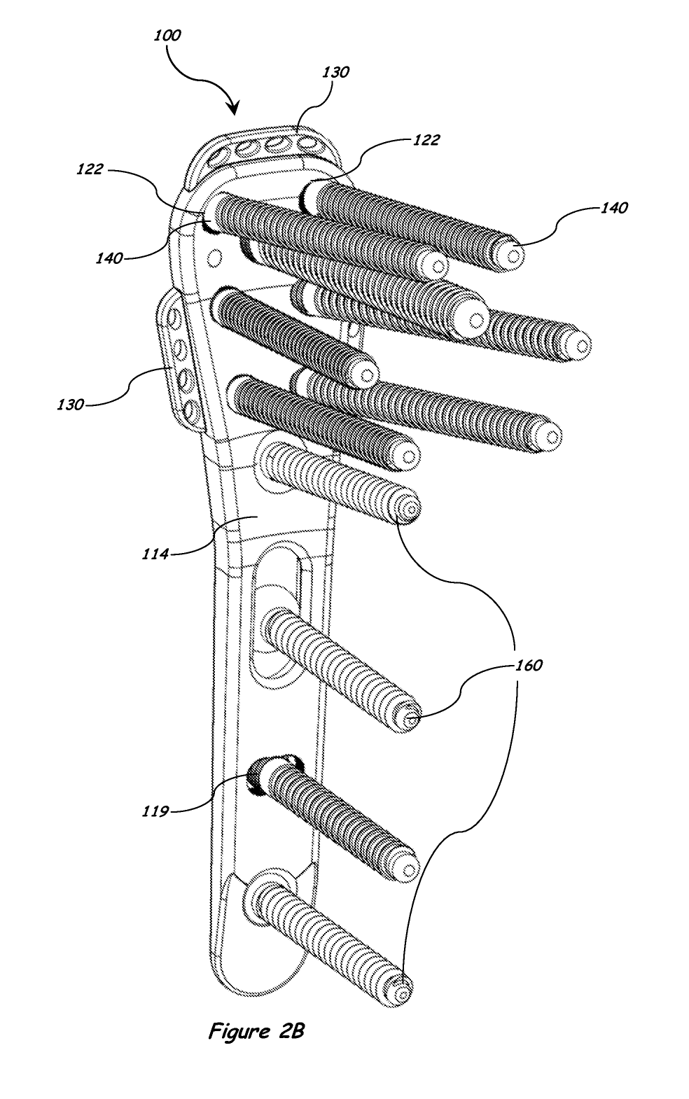

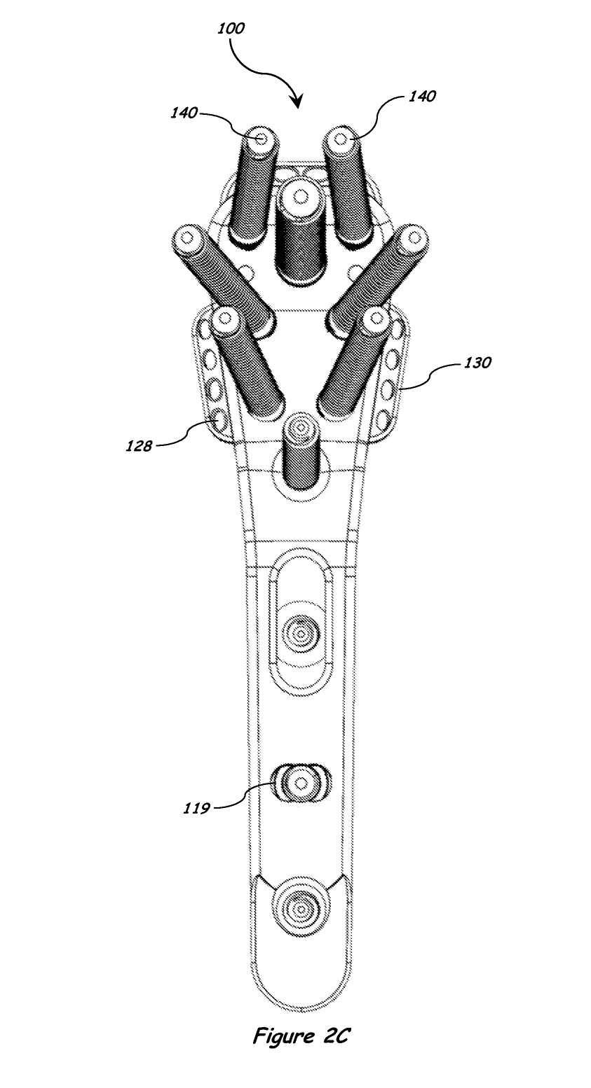

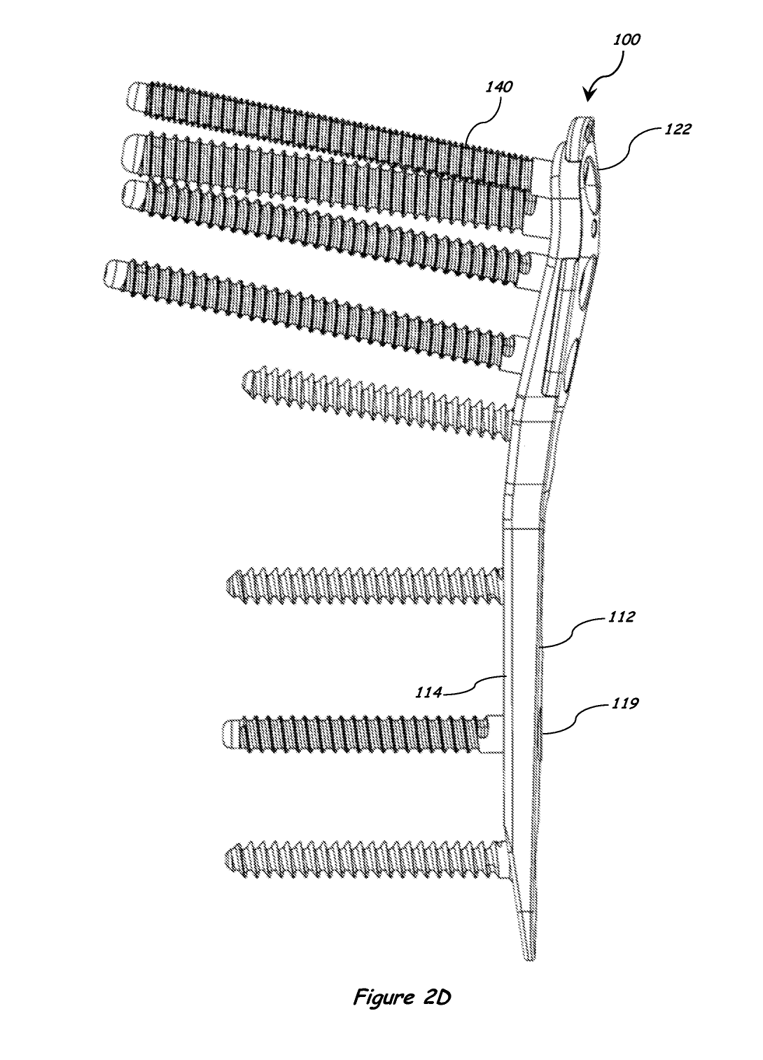

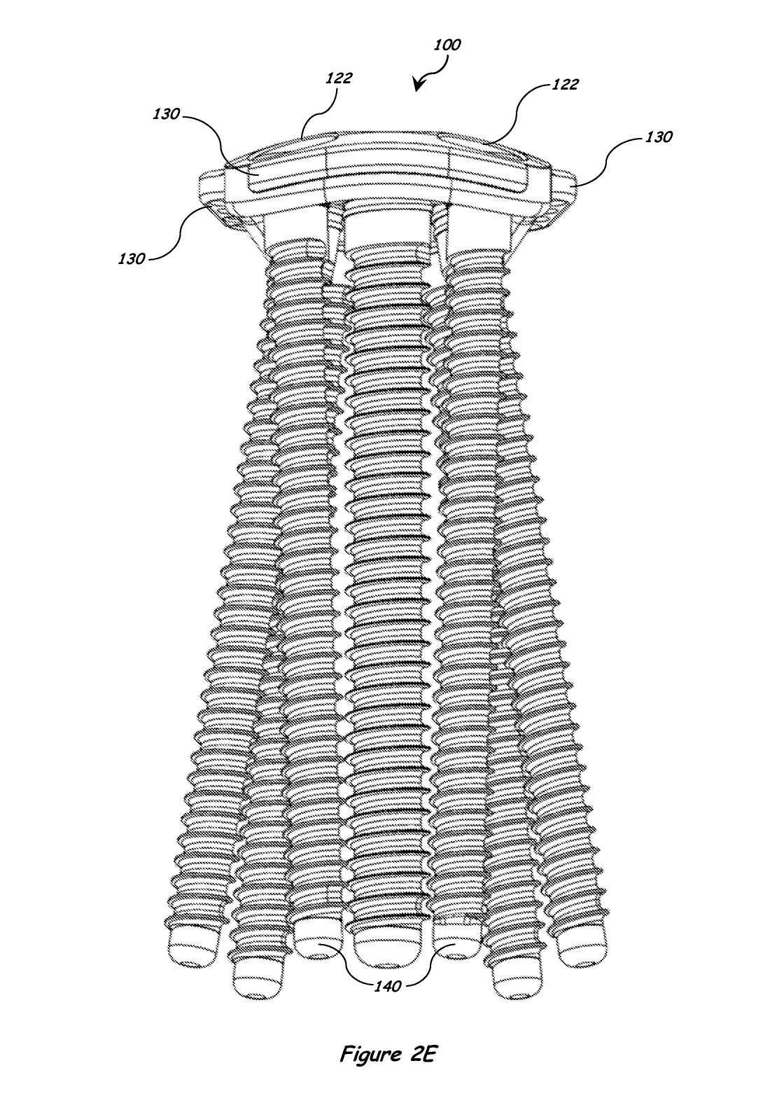

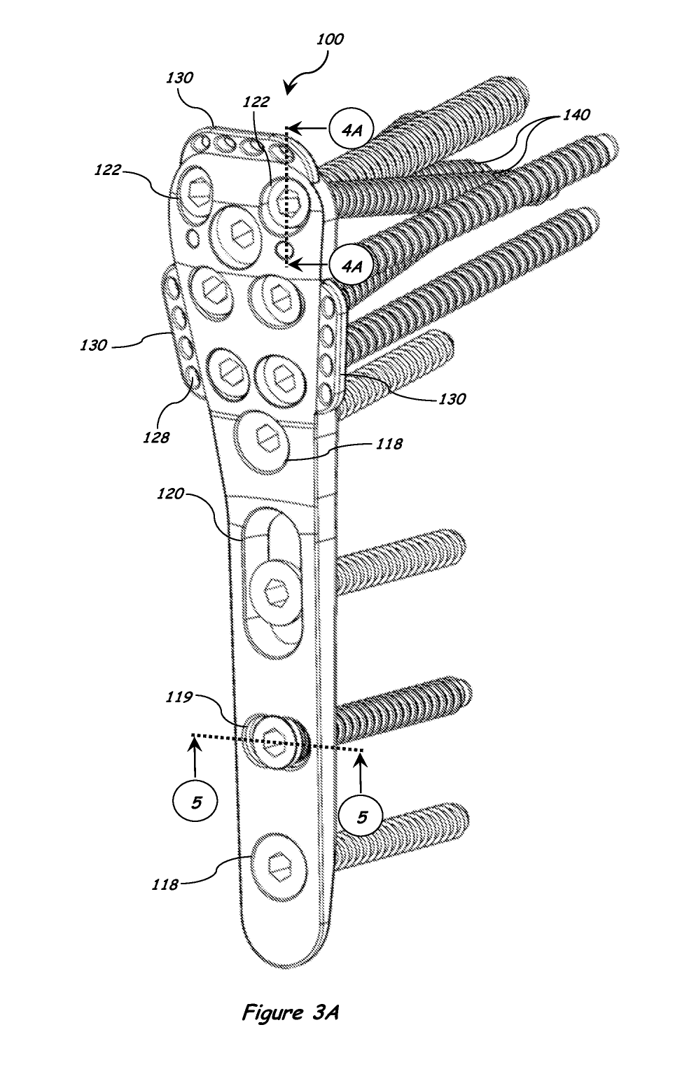

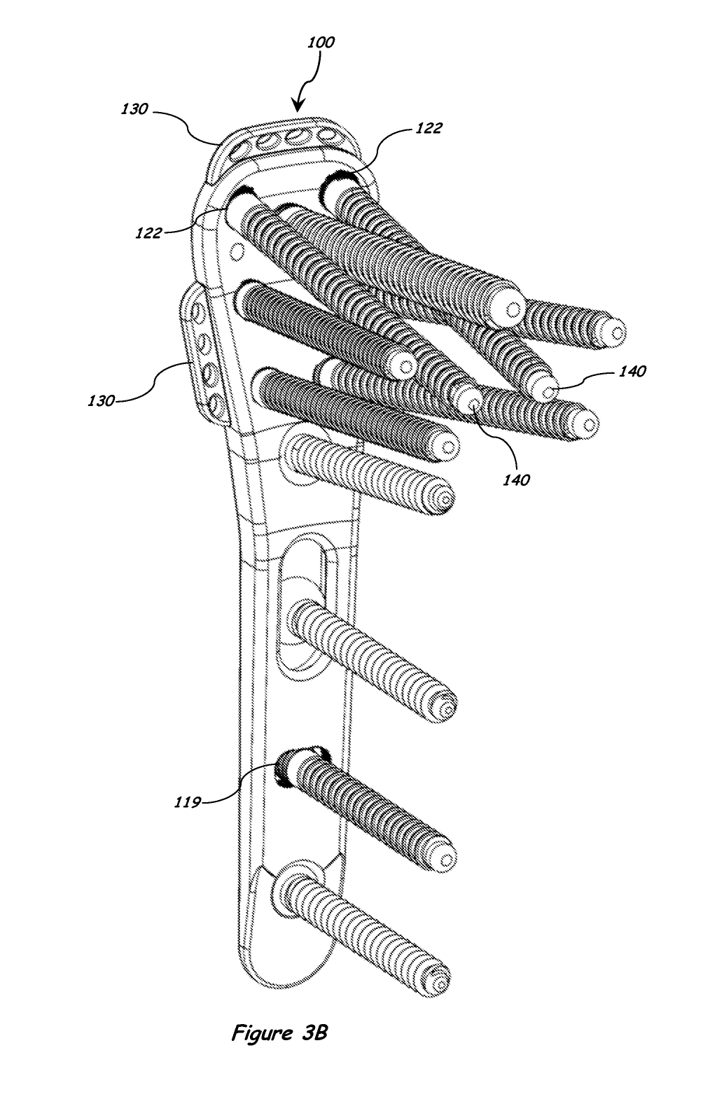

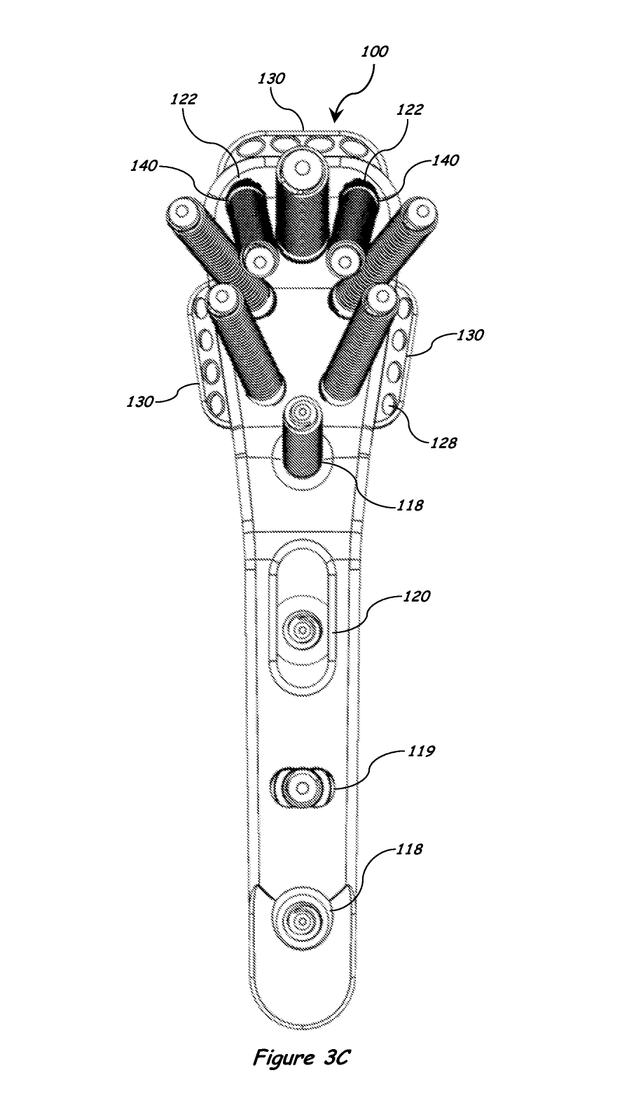

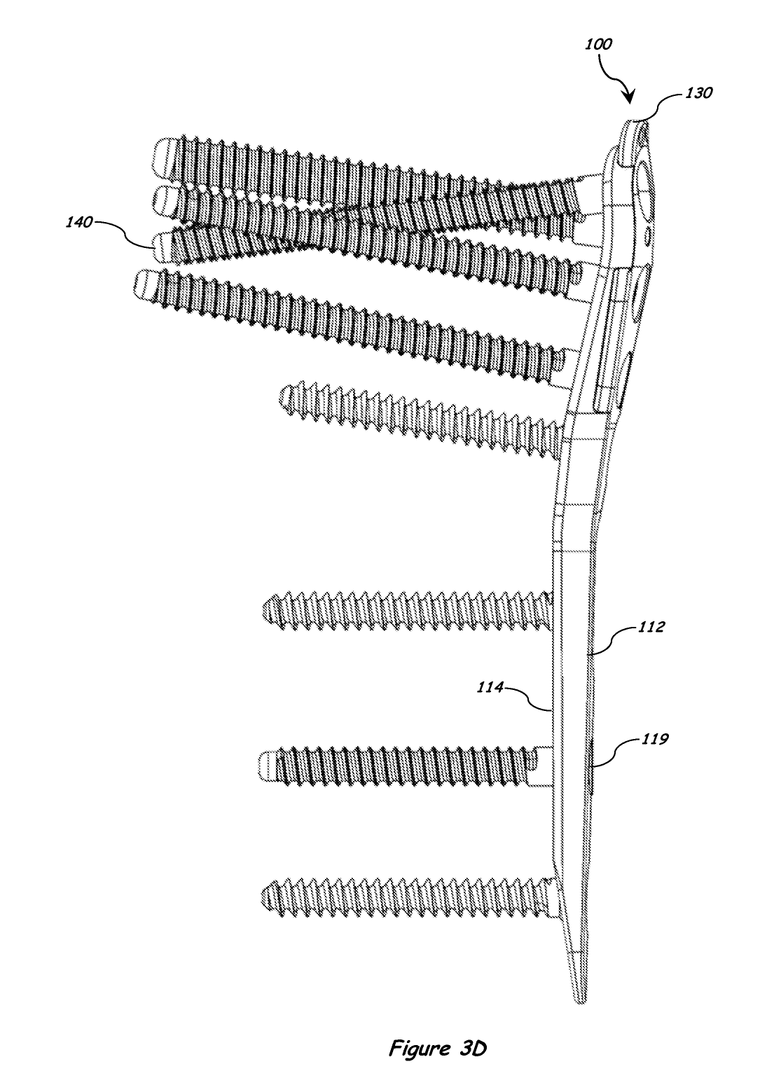

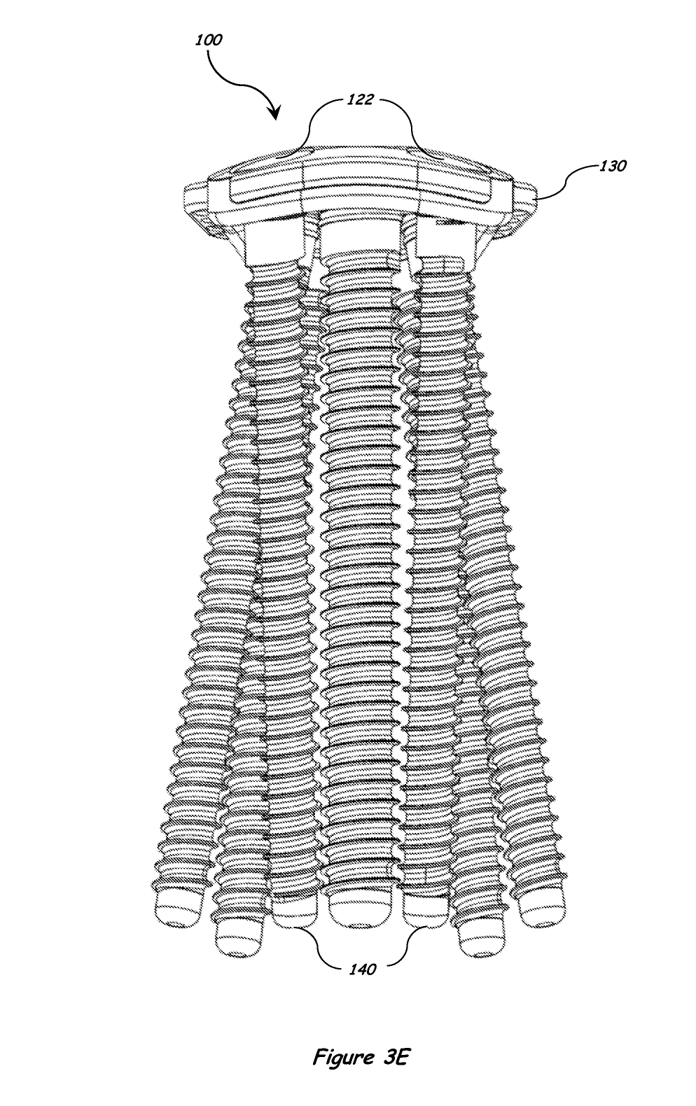

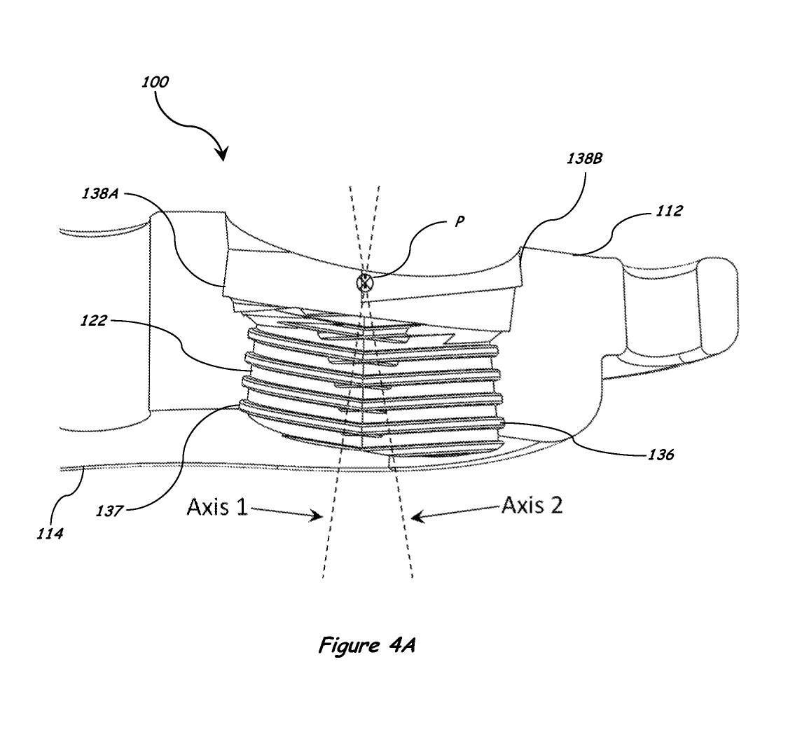

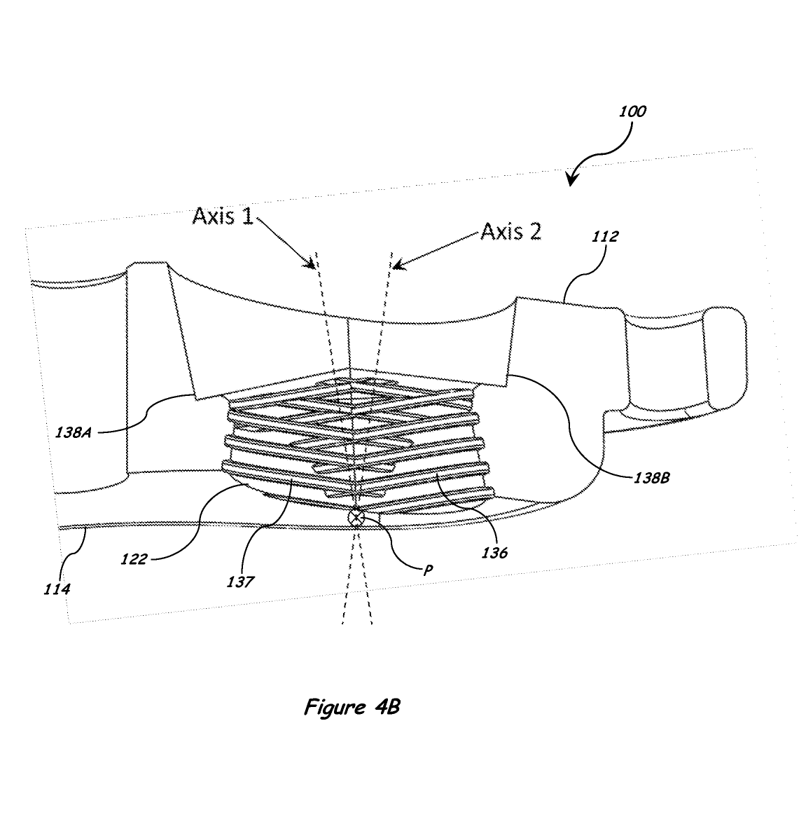

Referring to FIGS. 2A-E, 3A-E, bone plate 100 is configured substantially similar to bone plate 10 with screws 140, 160 inserted. Bone plate 100 comprises elevated suture hole structures 130 each includes a plurality of suture holes 128. The bone plate 100 is further provided with, but not limited to, two fastener holes 122 each formed with two sets of threads having intersecting axes, also referred to as bi-axial fastener holes in some embodiments. Where the axes cross is the intersect point or pivot point “P” which can be determined during manufacturing to be either within the fastener hole 122, or generally in the same plane as the upper surface 112 of the bone plate 100 as shown in FIG. 4A, or in the same plane as the bone-facing surface 114 of the bone plate 100 as illustrated in FIG. 4B. The provision of these bi-axial fastener holes 122 in the bone plate 100, particularly in the proximal or head portion of the bone plate 100, offers the surgeon with choice of two different and opposed trajectories, such as superiorly and inferiorly as illustrated in FIGS. 2A-E and FIGS. 3A-E respectively, for locking the bone fasteners 140 relative to the bone plate 100.

The two intersecting axes, axis 1 and axis 2 as illustrated in FIGS. 4A-B, may be configured to lie in a plane substantially parallel to a longitudinal plane dividing the bone plate into left and right halves, and form an angle a of about 5-45 degrees relative to one another. However, other angles are possible. In an alternative embodiment (not shown), the fastener holes 122 may include two sets of threads with intersecting axes lying on a plane substantially parallel to a transverse or cross-section plane dividing the bone plate into proximal and distal portions.

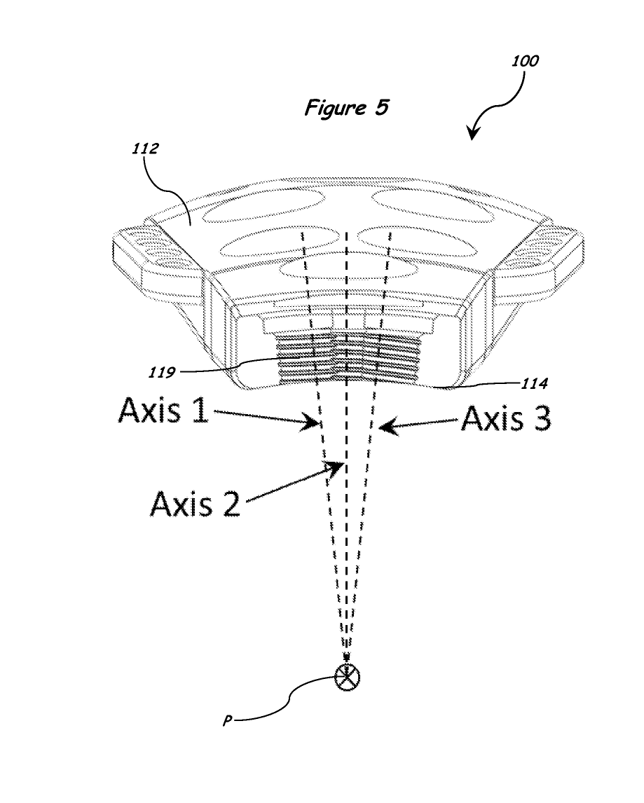

Additionally or alternatively, the bone plate 100 can be provided with one or more fastener holes, such as fastener hole 119 located in the distal portion of the bone plate 100, formed with three sets of threads with intersecting axes, such as axis 1, axis 2, axis 3 as shown in FIG. 5, wherein the intersect point or pivot point “P” of the axes is outside the fastener hole 119 and below the bone-facing surface 114 of the bone plate 100. These intersecting axes each form an angle o of about 5-45 degrees relative to one another. However, other angles are possible.

The bi-axial and tri-axial fastener holes 122, 119 can be formed in one of two methods. Referring to FIGS. 4A-B, the bi-axial hole 122 may be formed by drilling a hole along axis 1 at a predetermined angle relative to the bone plate 100 and another hole along axis 2 at a different predetermined angle relative to the bone plate 100 so that axis 1 and axis 2 intersect at a point “P” proximate the upper surface 112 of the bone plate 100, or proximate the bone-facing surface 114 of the bone plate 100. The countersinks 138A, 138B may be formed during or after drilling the holes. The threads 136, 137 may be right-hand threads and cut out with a machine tap that follows the individual axes of the respective drilled holes. The bi-axial hole, such as fastener hole 122, may have, but is not limited to, an oval or elongated shape from a top view of the fastener hole 122. The shape and size of the bi-axial holes 122 may vary throughout the thickness of the bone plate 100. Alternatively, the threads 136, 137 of the bi-axial hole 122 may not extend all the way from the upper surface 112 to the bone-facing surface 114 of the bone plate 100. A smooth, non-threaded, conical inward or outward taper may be formed into the upper or lower region of the bi-axial hole 122 (not shown) to provide for a broader range of angles for angularly positioning a non-locking bone fastener.

The tri-axial hole 119 as illustrated in FIG. 5 can be formed by drilling three separate holes through the bone plate 100 such that the hole axes intersect at a point below the bone facing surface 114 of the bone plate 100. The method of forming the countersinks and the threads may be similar to that of forming the bi-axial fastener hole 122.

Different types of screws/pegs may be used with the bi-axial and tri-axial holes 122, 119, including non-locking, locking, unidirectional and omnidirectional or surgeon-directed screws. One type of screw may be a locking screw that has a conically-tapered or cylindrical threaded head such as bone screws 140. The external threads of heads of the screws 140 may mate with the internal threads 136, 137 of the holes 122, 119 to angularly lock the screws/pegs 140 while the helical threads of the shaft of the screws/pegs 140 engage the bone.

The bone plate 100 further includes a non-threaded elongated slot 120 configured and dimensioned to engage a substantially spherical or hemi-spherical screw-head of a bone screw, such as bone screw 160. Alternatively, a conically shaped screw head, with or without threads, may engage the elongated slot 120. The elongated slot 120 may have a concave, substantially spherical portion or recess that opens toward the upper surface 112 of the bone plate 100. When the shaft of a bone screw 160 having a spherical or semi-spherical head is located eccentrically in the elongated slot 120, the spherical or semi-spherical head may engage the recess and bias the bone plate to provide compression of the bone fracture. The bone plate 100 may be provided with other non-locking, locking and/or combination holes for specific surgical applications.

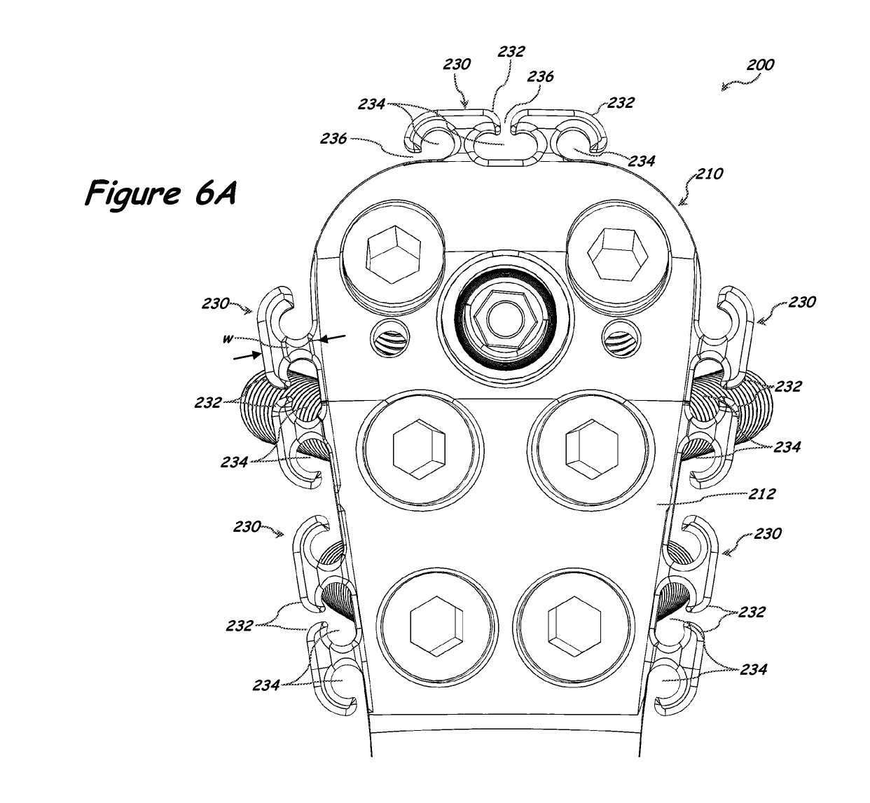

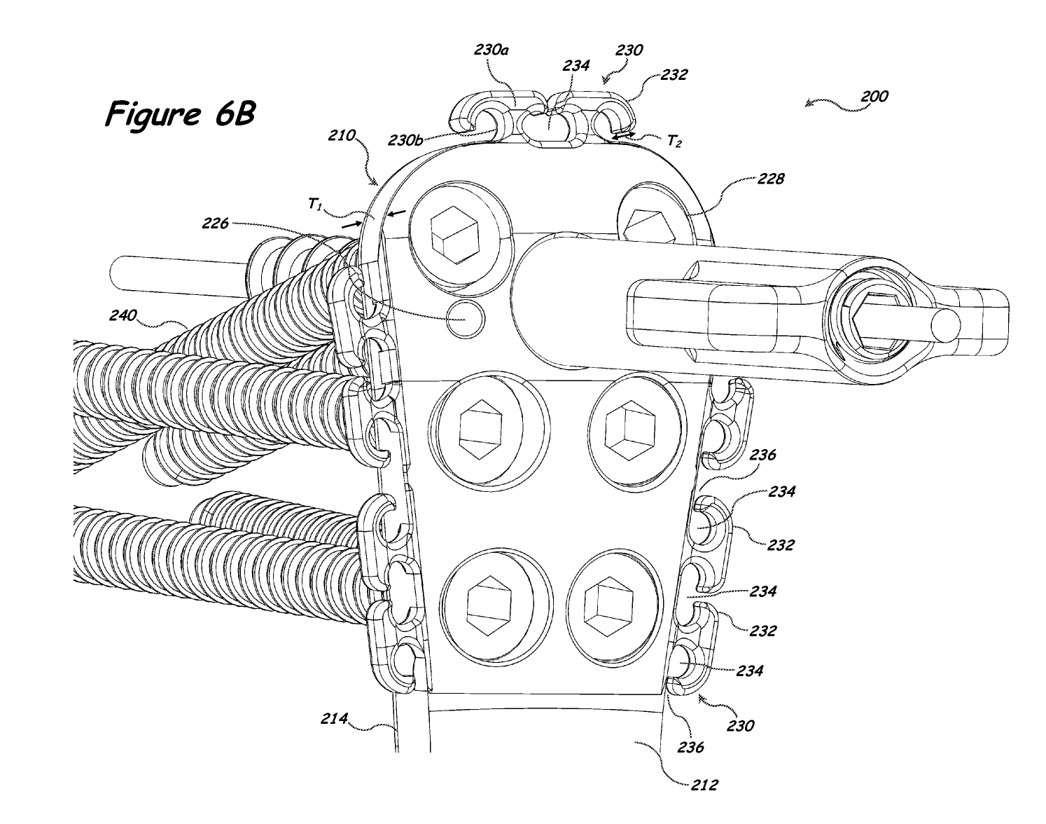

As shown in FIGS. 6A-B, a bone-fixation system 200 according to another exemplary embodiment of the invention comprises a bone plate, e.g. humeral fracture bone plate 210, formed with a plurality of fastener holes 228 and one or more k-wire holes 226 extending therethrough from an upper surface 212 to a bottom bone-facing surface 214 for receiving corresponding bone fasteners 240 and guide-wires respectively. The fastener holes 228 can include threaded, non-threaded, or a combination thereof, and optionally one or more bi-axial 122 or tri-axial 119 fastener holes discussed above. The bone-facing surface 214 can be shaped to substantially conform to or mate with a corresponding plate-facing surface of the bone (not shown) and may be provided with radiused or scalloped cutouts between fastener holes 228. Bone plate 210 may have various sizes (various dimensions, widths and/or lengths) and may be constructed from biocompatible materials such as titanium, alloys of titanium, cobalt chrome, stainless steel, ceramics, composite materials such as carbon fiber-reinforced PEEK, resorbable materials, and combinations thereof, although one of ordinary skill in the art will know and appreciate that any biocompatible material may be used.

As illustrated in FIGS. 6A-B, the upper surface 212 and the bone-facing surface 214 run substantially parallel defining a first nominal thickness “T₁” of the bone plate 210. The bone plate 210 further includes a plurality of suture cleats 230 extending from at least a portion of a boundary or edge of the bone plate 210. Suture cleats 230 each include protrusions or branches 232 forming open-ended recesses 234 having openings 236 being opened to different directions that the surgeon can easily and quickly tie a suture to or wrap a suture around to help stabilize and repair soft tissues, e.g. ligaments or tendons. As depicted in FIGS. 6A-B, the suture cleats 230 each have three open-ended recesses 234 including one middle or inner open-ended recess and two outer open-ended recesses that align substantially along the edge of the bone plate 210. The inner open-ended recess 234 has an opening 236 being located further away from the edge of the bone plate 210 than the openings 236 of the two outer open-ended recesses. The size and shape of the open-ended recesses 234 and openings 236 may vary to accommodate different sizes and turns of suture, suture guide and specific applications. Exemplary shapes of the open-ended recesses 234 include C-shape, U-shape, L-shape and V-shape.

Each suture cleat 230 includes an upper surface 230a and a lower bone-facing surface 230b defining a second nominal thickness “T₂” of the suture cleat 230 wherein the thickness “T₂” may be less than the thickness “T₁” of the bone plate 210, and the bone-facing surface 230b or at least a portion thereof of the suture cleat 230 is elevated above the bone-facing surface 214 of the bone plate 210 by a distance T₁-T₂ when the upper surface 212 of the bone plate 210 is flush with the upper surface 230a of the suture cleat 230. The distance T₁-T₂ may be in the range of about 1-5 mm. Alternatively, the distance T₁-T₂ can be lesser or greater, depending on a specific surgical application. When the upper surface 212 of the bone plate 210 is set to be above or below the upper surface 230a of the suture cleat 230, the bone-facing surface 230b or at least a portion thereof of the suture cleat 230 is elevated above the bone-facing surface 214 of the bone plate 210 by a distance lesser or greater than T₁-T₂ respectively.

The suture cleats 230 can be dimensioned and configured to provide a low profile for reducing soft tissue irritation and minimizing patient discomfort while offering needed strength and flexibility for specific surgical applications. The suture cleats 230 can be pliable and bendable to enable easy reshaping of the suture cleats 230 prior to or during operation. The suture cleats 230 may have a width “W” of about 0.5-4.5 mm. The width “W” may also be lesser or greater, depending on a specific surgical application. The upper surface 230a and the lower bone-facing surface 230b of the suture cleats 230 may be substantially parallel, at an angle relative to one another, or tapered inwardly and/or outwardly along its length or width. The upper surface 230a and the lower bone-facing surface 230b of the suture cleats 230 each may also have a convex or concave shape, or a combination thereof.

The upper surface 230a of the suture cleats 230 may be flush with the upper surface 212 of the bone plate 210. Alternatively, the upper surface 230a of the suture cleats 230 can be set slightly higher or lower than the upper surface 212 of the bone plate 210 and/or can have a substantially similar contour to a contour of the portion of the boundary or edge of the bone plate 210 from which the suture cleats 230 extends. The suture cleats 230 may be at an angle with respect to the bone plate 210. The suture cleats 230 each may have at least a portion of its body being as thick or thicker than the bone plate 210 so long as at least a portion of the bone-facing surface 230b of the suture cleats 230 is elevated above the bone-facing surface 214 of the bone plate 210 to provide a suture-clearance or spacing between the suture cleats 230 and the bone for easy access to the suture cleats 230.

The bone plate 210 can be formed with one or more suture cleats 230 integrally molded or separately affixed to the edge of the bone plate 210. In some other embodiments (not shown), the suture cleats may be formed in the body of the bone plate 210, e.g. the suture cleats located inside the boundary of the bone plate 210 and not necessary extending from the boundary or edge of the bone plate 210. The suture cleats 230 each may include all open-ended recesses 234 or a combination of one or more open-ended recesses 234 and one or more closed-ended loops or circular holes. The suture cleats 230 each may comprise a single or multiple separate or connected branches 232 forming any number of open-ended recesses as may be suitable for a specific surgical application. Alternatively, the suture cleats 230 each may have only one open-ended recess. The bone plate 210 can also be formed with a suture cleat 230 having all open-ended recesses 234 or a combination of one or more open-ended recesses 234 and one or more closed-ended loops or circular holes, and another suture cleat having all closed-ended loops, circular holes or combination thereof. Suture cleats with other designs and configurations that are known to one skilled in the art, e.g. boat cleats, are within the scope of the invention.

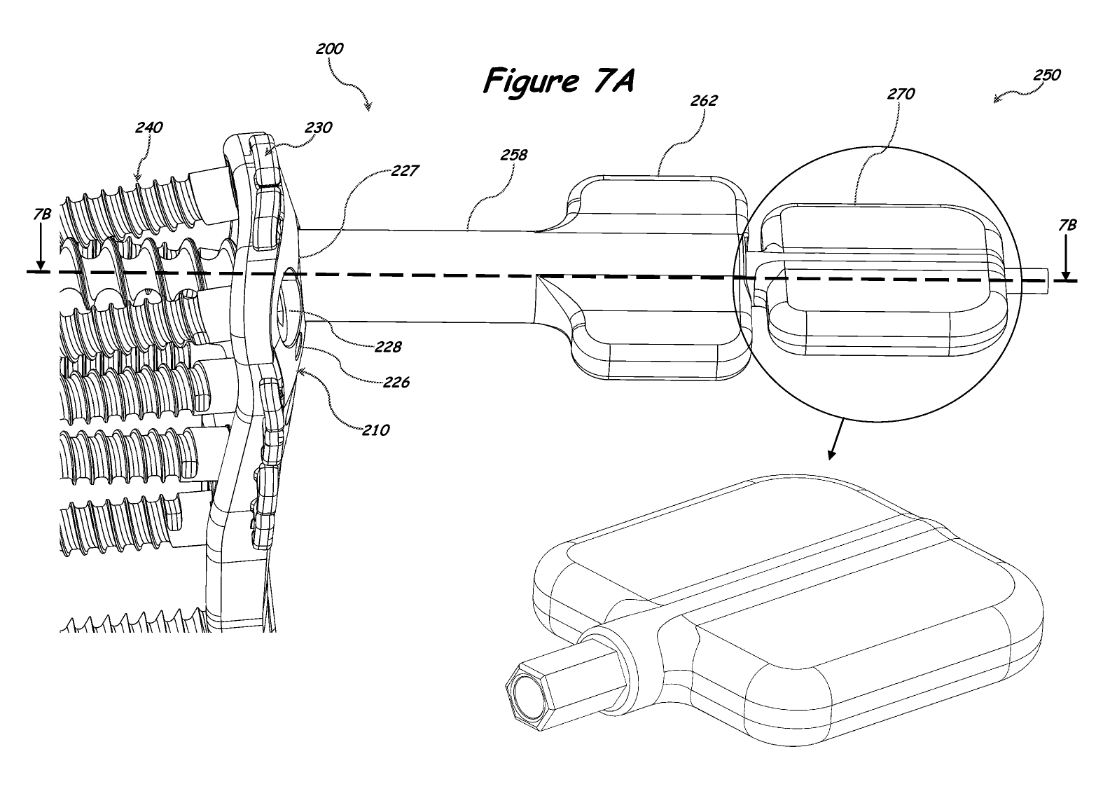

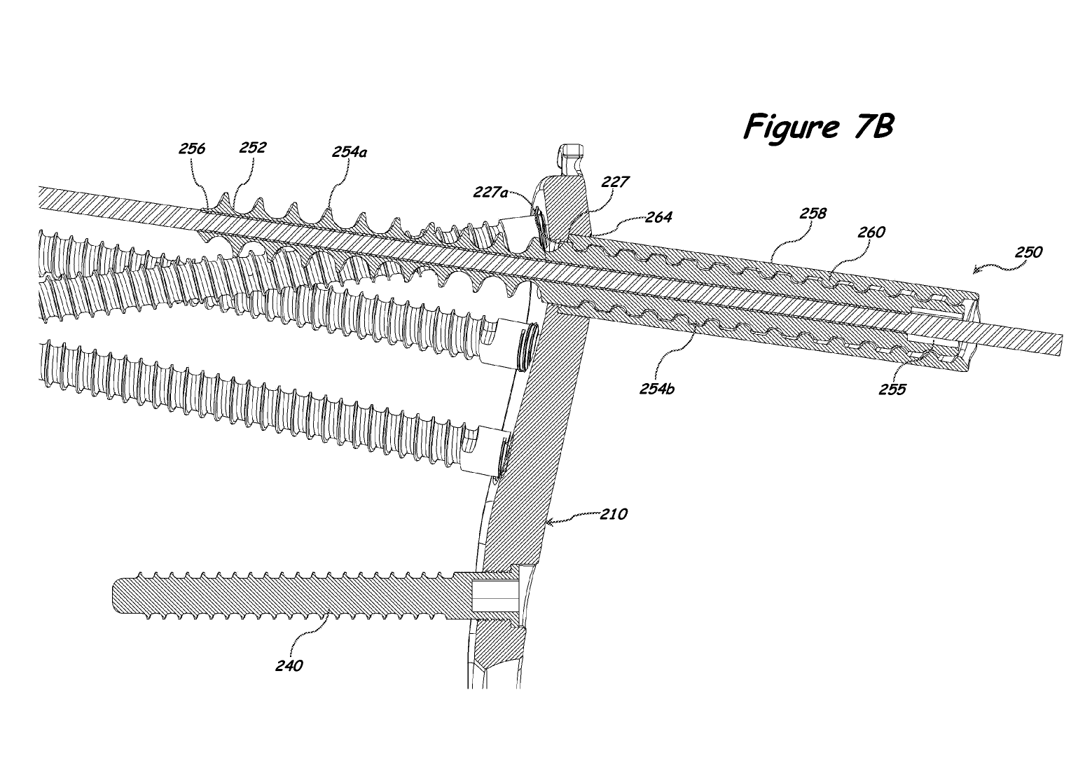

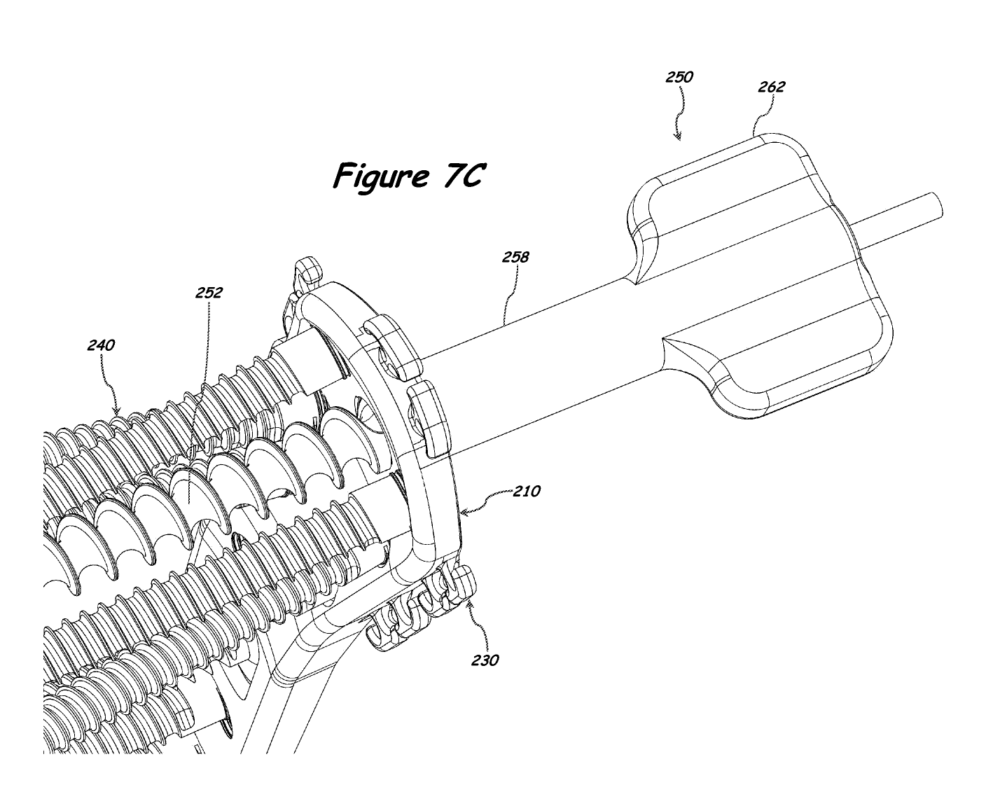

Referring now to FIGS. 7A-C, the bone fixation system 200 further includes an instrument or tool, e.g. plate compression device 250, for exerting a compression force on the bone plate 210 against a bone, e.g. humerus (not shown) to maximize the contact between the bone plate 210 and the bone prior to securing the bone plate 210 to the bone with the fasteners 240, and thus, to allow for a more stable construct. The plate compression device 250 includes a post component having a threaded exterior surface and a sleeve component having a threaded interior surface configured to threadedly engage with at least a portion of the threaded exterior surface to perform compression. The post component, by way of example without limitation thereto, compression screw 252, comprises cancellous threads, such as helical threads 254a, located on its proximal portion and adapted to provide a secure bone purchase, e.g. cancellous bone of the humeral head, and machine threads, such as helical threads 254b, located on its distal portion and configured to mate with internal threads of the corresponding sleeve component, by way of example without limitation thereto, compression nut 258. Compression nut 258 includes threaded interior surface 260 configured to interface with at least a portion of the threaded exterior surface of the post component, e.g. helical threads 254b, to perform compression. The external cancellous threads 254a and machine threads 254b can be made to have substantially the same or different thread spiral, pitch and tooth profile as known to one skilled in the art to perform the compression effectively. The post component may include a longitudinal inner through bore 256 for receiving a guide wire/pin. Optionally, the distal end of the post component, e.g. compression screw 252, may include a driver engagement feature, such as hexagonal socket 255, adapted to receive a driver, by way of example, hexagonal driver 270, to aid in driving the compression screw 252 into a bone, and also to keep the compression screw 252 stationary when needed while compression nut 258 is rotated on the compression screw 252 to provide required compression between the bone plate 210 and a bone. The sleeve component, e.g. compression nut 258 may include a handle 262 for manipulation by a user on its distal end and a plate engagement end 264 on its proximal end.

As depicted in FIG. 7A, bone plate 210 may include a plate interface hole 227 formed therethrough extending from an upper surface 212 to a bottom bone-facing surface 214. The plate interface hole 227 may include one or more protrusions 227a integrally formed on its inner walls to engage with the plate engagement end 264 of the compression nut component 258 to exert compression forces on the bone plate 210.

In another aspect, the present invention is directed to a method of performing a surgery, e.g.

bone fracture fixation, using the bone plate 210 having suture cleats 230 discussed above. An exemplary procedure for repairing a proximal humeral fracture using the invention bone plate with suture cleats 210 and the plate compression device 250 includes the following steps: after proximal humerus bone fragments are approximated and held in place with k-wires, heavy sutures may be used and attached to the tendon-bone interface of the rotator cuff muscles and placed to the side. Bone plate 210 may then be attached to humeral shaft by placing a non-locking screw in an oblong hole or slot (not shown) in the bone plate 210 and into the bone and adjusting the plate position just lateral to the biceps groove. A provisional guide pin or wire may be inserted through the plate interface hole 227 and imaged with fluoroscopy to ascertain correct placement into humeral head. Compression screw 252 of the plate compression device 250 may be placed over the provisional guide pin and through the plate interface hole 227 and threaded into the humeral head using a driver, such as hexagonal screwdriver 270, so it bisects the humeral head fragment. While keeping compression screw 252 stationary using the hexagonal driver 270, compression nut 258 may then be rotated on compression screw 252 until it contacts the bone plate 210. Additional rotation of compression nut 258 may be performed until humeral head fragment is compressed against the underside of bone plate 210. Finally, locking screws/pegs and shaft screws may be inserted in a divergent pattern through respective fastener holes in the bone plate 210 to lock the bone plate 210 and stabilize the bone fragments. After the installation of the bone plate 210, the plate compression device 250 is removed and may be replaced with a locking cancellous screw in the plate interface hole 227. Next, the sutures from rotator cuff muscles may be attached to the suture cleats 230 of bone plate 210 prior to closing the incision. An exemplary method for tying the suture to the suture cleats 230 includes the steps of placing suture through the middle recess or slot of suture cleat 230, wrapping suture around suture cleat 3-4 times using a figure eight pattern, forming a loop on an end of the suture and spin the loop one time, place the loop on opposite side cleat and pull on suture to tighten loop, repeat steps of forming and tightening a loop two or more times, then tie off remaining suture to cleats by using multiple slip ties, and cut off excess suture.

The bone plate system of the present invention provides for any tissue repair and attachment of soft tissue to bone as part of fracture management repair. Although there have been described and illustrated herein various embodiments of a humeral fracture bone plate, it is not intended that the invention be limited thereto, as it is intended that the invention be as broad in scope as the art will allow and that the specification be read likewise. Thus, while the exemplary embodiment is described and illustrated as a humeral fracture bone plate system, it is appreciated that the system is well adapted to bone fractures of any bones with or without an articular convex-shape surface. Thus, the system of the invention could similarly be used to treat fracture of other bones, e.g., a fracture of the femoral head, a fracture of a radius. In addition to the use of the present bone plate system for treatment of fractures, it is appreciated that the present invention may also be used in the treatment of osteotomies and non-unions of the proximal humerus and other bones with or without an articular convex-shaped surface. Furthermore, the invented suture cleats could similarly be used on other orthopedic implants for repairing soft tissue, bone fracture or a joint, by way of example without limitation thereto, a bone screw or fastener or peg, a fracture or suture plate, a nail, a joint prosthesis (e.g. hip, knee or shoulder prosthesis) or any combinations thereof.

The principles, preferred embodiments and modes of operation of the present invention have been made apparent in the foregoing description.

Although the embodiments are numbered with, for example, “first,” “second,” or “third,” or “fourth,” the ordinal numbers do not imply priorities of the embodiments.

Since many modifications, variations and changes in detail can be made to the described embodiments of the invention, it is intended that all matters in the foregoing description and shown in the accompanying drawings be interpreted as illustrative and not in a limiting sense. Thus, the scope of the invention should be determined by the appended claims and their legal equivalents.

1. A bone fixation system for securing a bone plate to a bone, the system comprising:
a bone plate formed with at least one fastener hole extending therethrough from a first upper surface to a first lower bone-facing surface, said bone plate comprises at least one suture cleat including one or more branches forming one or more open-ended recesses; and
a bone fastener adapted to couple the bone plate to the bone.

2. The bone fixation system of claim 1, wherein the at least one suture cleat extends from at least a portion of an edge of the bone plate.

3. The bone fixation system of claim 2, wherein the at least one suture cleat comprises a plurality of open-ended recesses having openings being opened to different directions.

4. The bone fixation system of claim 2, wherein the at least one suture cleat has a suture cleat thickness defined by a second upper surface and a second lower bone-facing surface, and at least a portion of the second bone-facing surface of the suture cleat is elevated above the first bone-facing surface of the bone plate by a distance of greater than 0 mm.

5. The bone fixation system of claim 4, wherein the suture cleat thickness is less than a thickness of the bone plate.

6. The bone fixation system of claim 3, wherein the at least one suture cleat includes at least one closed-ended loop or circular hole.

7. The bone fixation system of claim 4, wherein the second bone-facing surface of the suture cleat is elevated above the first bone-facing surface of the bone plate by a distance of less than about 5 mm.

8. The bone fixation system of claim 3, wherein the at least one suture cleat is integrally formed or separately affixed to the edge of the bone plate.

9. The bone fixation system of claim 3, wherein the at least one suture cleat has a width in the range of about 0.5-4.5 mm.

10. The bone fixation system of claim 3, wherein the second bone-facing surface of the at least one suture cleat is substantially parallel to or at an angle with respective to the first bone facing surface of the bone plate.

11. The bone fixation system of claim 2, wherein at least one of the bone plate, the at least one suture cleat and the fastener is constructed from titanium, titanium alloys, stainless steel, tantalum, composite materials, resorbable materials, biocompatible materials or combinations thereof.

12. The bone fixation system of claim 2, wherein the fastener hole includes a plurality of sets of threads with intersecting axes.

13. The bone fixation system of claim 12, wherein the intersecting axes of the plurality of sets of threads lie in a plane substantially parallel to at least one of a longitudinal plane which divides the plate into left and right portions and a transverse plane which divides the bone plate into proximal and distal portions.

14. The bone fixation system of claim 13, wherein the bone fastener comprises a head having a thread for forming a threaded connection with at least one of the sets of threads of the fastener hole.

15. The bone fixation system of claim 14, wherein the bone plate further comprises a second fastener hole having at least three sets of threads with intersecting axes.

16. The bone fixation system of claim 12, wherein the intersecting axes form an angle of about 5 to 45 degrees relative to one another.

17. The bone fixation system of claim 1, wherein the bone is one of humerus, ulna, radius, clavicle, femur, tibia, fibula, tarsals, metatarsals, carpals, metacarpals and phalanges.

18. A bone fixation system for securing a bone plate to a bone, the system comprising:
a bone plate formed with at least one fastener hole extending therethrough from a first upper surface to a first lower bone-facing surface;
at least one suture cleat extending from at least a portion of an edge of the bone plate and comprising one or more branches forming at least three open-ended recesses including one inner open-ended recess and two outer open-ended recesses aligned substantially along the edge portion of the bone plate;
wherein the inner open-ended recess has an opening located further away from the edge portion of the bone plate than openings of the outer open-ended recesses; and
a bone fastener adapted to couple the bone plate to the bone.

19. A method of performing surgery comprising:
providing a bone plate formed with at least one fastener hole extending therethrough from an upper surface to a lower bone-facing surface, said bone plate comprises at least one suture cleat extending from a portion of an edge of the bone plate and including one or more branches forming one or more open-ended recesses;
providing a bone fastener adapted to couple the bone plate to a bone;
providing a suture;
securing the bone plate to the bone with the bone fastener; and
stabilizing a soft tissue by attaching the soft tissue to the suture and wrapping the suture around the at least one suture cleat.

20. The method of claim 19 further comprising:
providing a plate interface hole in the bone plate;
providing a plate compression device including:
a post component having threaded exterior surface; and
a corresponding sleeve component having threaded interior surface configured to interface with the threaded exterior surface to perform a compression; and
compressing the bone plate against the bone prior to the step of securing the bone plate, the step of compressing including:
passing the post component through the plate interface hole and into the bone; and
rotating the sleeve component on the post component until the sleeve component contacts a portion of the bone plate to exert compression forces on the bone plate against the bone.

21. An orthopedic implant comprises at least one suture cleat including one or more branches forming one or more open-ended recesses.

22. The orthopedic implant of claim 21, wherein the at least one suture cleat is integrally molded with or separately affixed to the orthopedic implant.

23. The orthopedic implant of claim 22 comprises one of a bone fastener, a bone plate, a suture plate, an intramedullary nail, and a joint prosthesis.

24. The orthopedic implant of claim 23, wherein the joint prosthesis comprises one of a hip prosthesis, a knee prosthesis, and a shoulder prosthesis.
